(19) United States
(12) Patent Application Publication
(10) Pub. No.: US 2023/0384215 A1
(43) Pub. Date: Nov. 30, 2023

(54) MULTILAYER ENVIRONMENT SCANNING

(71) Applicant: DeepX Diagnostics, Inc., Miami, FL (US)

(72) Inventors: Jakob J. Stamnes, Oslo (NO); Knut Stamnes, Hoboken, NJ (US); Børge Hamre, Sandsli (NO); Snorre Stamnes, Yorktown, VA (US); Yongzhen Fan, Rockville, MD (US); Nan Chen, Denver, CO (US)

(21) Appl. No.: 18/204,213

(22) Filed: May 31, 2023

Related U.S. Application Data
(60) Provisional application No. 63/347,409, filed on May 31, 2022.

(51) Int. Cl. G01N 21/31

(52) U.S. Cl. CPC G01N 21/31

(57) ABSTRACT

A method for remote sensing of a multilayer environment emits a radiance signal toward a first layer and a second layer of the-multilayer environment, at least one of the first layer or the second layer being optically thick. The method receives the radiance signal including a first component corresponding to the first layer, and a second component corresponding to the second layer. The method then determines the second component based on the radiance signal.

### PRIORITY

This patent application claims priority from provisional U.S. patent application No. 63/347,409, filed May 31, 2022, entitled, “TISSUE DIAGNOSTICS BASED ON OPTICAL TRANSFER DIAGNOSIS,” and naming Jakob Stamnes et al. as inventors, the disclosure of which is incorporated herein, in its entirety, by reference.

### FIELD

Illustrative embodiments of the invention generally relate to remote sensing applications and, more particularly, various embodiments of the invention relate to sensing properties of different layers in multilayered environments.

### BACKGROUND

Remote sensing may include observing electromagnetic radiation interacting with an environment. For example, a sensing system may determine characteristics of the environment based on attributes of the electromagnetic radiation before and after the electromagnetic radiation interacts with layers of the environment. Existing systems may attribute the characteristics to the environment as a whole even though the characteristics may only be valid for portions of the environment.

### SUMMARY OF VARIOUS EMBODIMENTS

In accordance with one embodiment, a method for remote sensing of a multilayer environment emits a radiance signal toward a first layer and a second layer of the multilayer environment. The method receives the radiance signal including a first component corresponding to the first layer, and a second component corresponding to the second layer. The method determines the second component based on the radiance signal. At least one of the first layer or the second layer is optically thick.

In some embodiments, the method outputs a spectral reflectance image using the determined second component. Emitting the radiance signal may include emitting a collimated beam.

The first layer may have at least one of a different refractive index, a different absorption characteristic, or a different scattering characteristic relative to the second layer. In some embodiments, the first layer is comprised of gaseous matter, and the second layer is comprised of soft matter. In some embodiments, a difference between the refractive indices of the first layer and the second layer is at least 0.3.

Determining the second component may include integrating a source function for the first layer.

In some embodiments, the method estimates radiance signal components using an environment model. Determining the second component may include updating an optical property of the second layer of the multilayer environment after comparing the estimated radiance signal components and the determined second component.

In accordance with another embodiment of the invention, a scanning system has a measuring circuit to emit a radiance signal toward a first layer and second layer of the multilayer environment and receive the radiance signal including a first component corresponding to the first layer and a second component corresponding to the second layer. The scanning system also has a layer data analysis circuit configured to determine the second component based on the received radiance signal. At least one of the first layer or the second layer of the multilayer environment is optically thick.

In some embodiments, the scanning system has an output circuit to output a spectral reflectance image using the determined second component. The measuring circuit emits the radiance signal by emitting a collimated beam.

The first layer may have at least one of a different refractive index, a different absorption characteristic, or a different scattering characteristic relative to the second layer. The first layer may be comprised of gaseous matter, while the second layer is comprised of soft matter. In some embodiments, a difference between the refractive indices of the first layer and the second layer is at least 0.3.

In some embodiments, the data layer analysis circuit estimates radiance signal components using an environment model. Determining the second component may include updating an optical property of the first layer and the second layer of the multilayer environment after comparing the estimated radiance signal components with the determined first and second components.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a scanning system in accordance with various embodiments.

FIG. 2 shows a process for remote sensing of a multilayer environment in accordance with various embodiments.

FIG. 3 schematically shows a computing system in accordance with various embodiments.

### DESCRIPTION OF ILLUSTRATIVE EMBODIMENTS

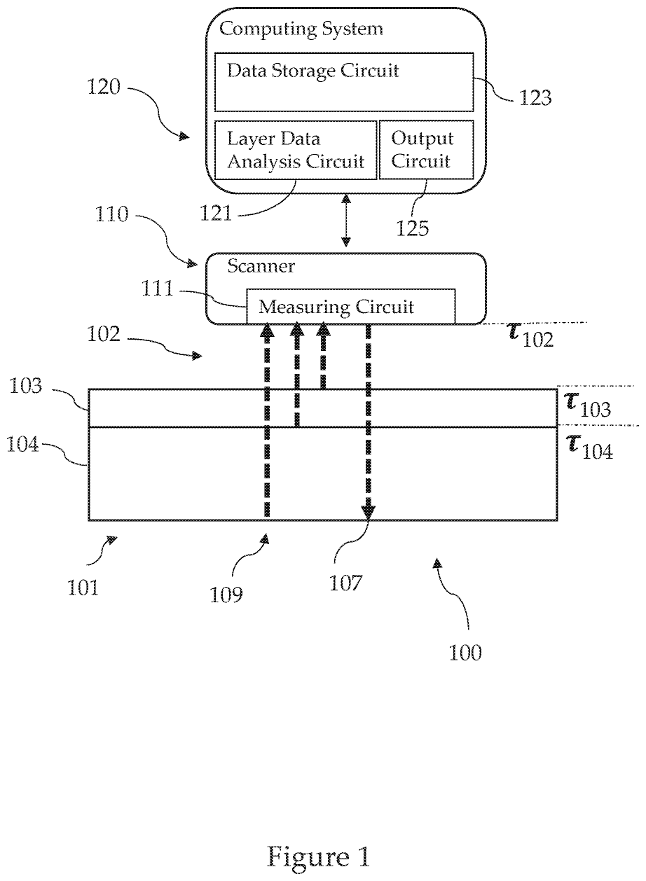

FIG. 1 schematically shows a scanning system 100 configured to scan a multilayer environment 101 and determine characteristics of at least one individual layer. Unlike other scanning systems, the scanning system 100 does not rely on gels or other media placed between the system 100 and the observed environment due to the scanning system 100 being configured to determine characteristics of a target layer irrespective of varying refractive indices in the environment 101.

In the illustrated embodiment, the multilayer environment 101 has layers 102, 103, and 104, each layer being adjacent to at least one other layer at a layer interface. The layers 102, 103, and 104 have varying thicknesses and refractive indices among other characteristics. The illustrated environment 101 is not intended as a limitation of the user of the scanning system 100. In other embodiments, the multilayer environment 101 may have more or fewer layers, and the layers may have different thicknesses and/or uniform thicknesses. The layers may have different refractive indices than the illustrated refractive indices. For example, layer 102 is comprised of air, having a real refractive index of 1.0, whereas layers 103 and 104 have a complex refractive index, with a real component of 1.34 and a varying imaginary component.

In the illustrated embodiment, the multilayer environment 101 represents an air-tissue environment where layer 102 is an air layer between the scanner 110 and the tissue layers 103 and 104 representing layers of skin. In other embodiments, the layers of the multilayer environment 101 may have a gaseous state, a liquid state, a solid state, or a plasma state, among other things. The layers may be comprised of any material. For example, another multilayer environment may include an atmospheric environment.

In some embodiments, the scanning system 100 may determine characteristics of different layers in the multilayer environment 101. These characteristics may be used to determine tissue abnormalities, among other things. For example, the layer characteristics may be used to detect skin cancer. Successive scans over time may monitor changes in the growth, size, color, or shape of an area of interest of the tissue. The scanning system 100 may also detect vertical growth through layers of tissue, such as the growth of melanocyte cells into the dermis layer, an indicator of skin cancer.

The scanning system 100 has a scanner 110 to emit and receive a radiance signal. The scanner 110 may be a handheld device, a portable device, or a fixture, among other things. As illustrated, the scanner 110 is positioned above the multilayer environment 101. In other embodiments, such as for atmospheric sensing, the scanner 110 may be located under the multilayer environment 101, among other things.

The scanner 110 has a measuring circuit 111 configured to emit a radiance signal 107 toward the multilayer environment 101 and receive the radiance signal 107 (comprised of components 109) from the multilayer environment 101. In some embodiments, the scanner 110 may include more than one device, and may be configured such that one device emits the radiance signal 107 and another device receives the radiance signal 107 (comprised of components 109).

The radiance signal 107 may include electromagnetic radiation. In some embodiments, the radiance signal 107 is emitted from the measuring circuit 111 by emitting a collimated beam. The radiance signal 107 transmitted by the scanner 110 may be a continuous signal or a pulsing signal, among other things.

The radiance signal 107 (comprised of components 109) received by the measuring circuit 111 has been altered from when the radiance signal 107 was emitted by the measuring circuit 111 through interaction with the multilayer environment 101. In the illustrated example, the radiance signal 107 returning to the measuring circuit 111 is comprised of components 109 (e.g., backscatter) corresponding to one of the layers of the environment 101.

The scanning system 100 also includes a computing system 120 to process the radiance signal 107 received by the scanner 110, among other things. In the illustrated embodiment, the scanner 110 and the computing system 120 are distinct; however, in other embodiments, the scanner and the computing system 120 may be fully or partially incorporated into the same device.

The computing system 120 has a data storage circuit 123 to store environment models as described below.

The scanning system 100 has a layer data analysis circuit 121 to determine layer characteristics based on the received radiance signal 107 and/or the environment models stored by the data storage circuit 123.

In some embodiments, the layer data analysis circuit is configured to estimate components of the radiance signal 107 corresponding to one or more layers of the multilayer environment 101, and compare the estimated components to the actual signal components.

The signal component for each layer may be determined using one of the following equations, where I is the Stokes vector, τ is the optical depth, μ is the cosine of the polar angle θ (cos θ), ϕ is the azimuthal angle, p and n are layer indices, S is the source function, and t is time.

$${I}^{+} ( τ , μ , ϕ ) = {\int }_{τ}^{{τ}_{p}} \frac{dt}{μ} {S}_{p}^{+} ( t , μ , ϕ ) {e}^{- ( t - τ ) / μ} + {\sum }_{n = p + 1}^{L} {\int }_{{τ}_{n - 1}}^{{τ}_{n}} \frac{dt}{μ} {S}_{n}^{+} ( t , μ , ϕ ) {e}^{- ( t - τ ) / μ} \qquad ( 1 )$$

$${I}^{-} ( τ , μ , ϕ ) = {\sum }_{n = 1}^{p - 1} {\int }_{{τ}_{n - 1}}^{{τ}_{n}} \frac{dt}{μ} {S}_{n}^{-} ( t , μ , ϕ ) {e}^{- ( τ - t ) / μ} + {\int }_{{τ}_{p - 1}}^{τ} \frac{dt}{μ} {S}_{p}^{-} ( t , μ , ϕ ) {e}^{- ( τ - t ) / μ} \qquad ( 2 )$$

Equations (1) and (2) integrate the source function layer by layer to yield the Stokes vector of the diffuse total and polarized radiance. The equations quantify the contributions from any specific layer in a multilayer environment.

The source function in equations (1) and (2) may be evaluated by the discrete ordinate method, among other methods, and therefore have analytic solutions.

The computing system 120 has an output circuit 125 configured to generate an output based on the determined radiance signal 107 component of layers of the multilayer environment 101. In some embodiments, the output circuit 125 outputs an updated model of the multilayer environment 101, or a portion thereof, based on the layer contributions determined using equations (1) or (2). In some embodiments, the output circuit generates a visual representation of a target layer based on the radiance signal 107 component of the target layer.

The scanning system 100 can evaluate characteristics of individual layers even if one of the layers is optically thick. Optical thickness may be understood to be the degree to which a layer prevents the radiance signal 107 from passing through. When a layer is optically thick, there is multiple scattering such that some backscatter components 109 are delayed relative to other components 109. A layer that is optically thick has a large optical depth and light is easily absorbed by the layer. In some embodiments, a layer is optically thick if the product of the thickness of the layer times the single-scattering albedo of the layer is greater than 0.05. In another embodiment, a layer is optically thick if the product of the thickness of the layer times the single-scattering albedo of the layer is greater than 0.1.

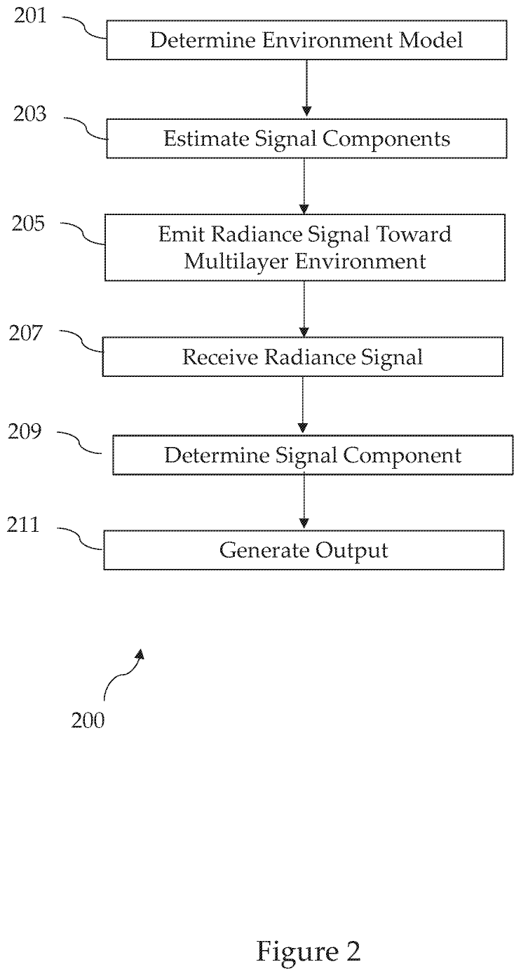

FIG. 2 shows an exemplary process 200 for remote sensing of a multilayer environment in accordance with the illustrated embodiment. In a preferred embodiment, the layers of the multilayer environments have different refractive indices. For example, the first layer may be air while the second layer may be soft matter such as living tissue from a human or other organism. In another example, the first layer may be air and the second layer may be paint. The process 200 may be implemented in whole or in part in the scanning system 100 disclosed herein. In certain forms, the operations may be performed by the scanner 110 and the computing system 120 of the scanning system 100. In certain forms, all functionalities may be performed by the same device, such as where the computing system 120 and the scanner 110 are incorporated into one device. It shall be further appreciated that a number of variations and modifications to the process 200 are contemplated including, for example, the omission of one or more aspects of the process 200, the addition of further conditionals and operations, or the reorganization or separation of operations and conditionals into separate processes.

The process 200 begins at operation 201 where the scanning system 100 determines an initial environment model. In some embodiments the scanning system 100 determines the initial model by retrieving the initial model from the data storage circuit 123. The initial model may be derived using historical scans of the patient, biopsies of the patient, a digital medical record of the patient, another type of medical record of the patient, or data sources corresponding to another patient or patients, among other things. In some embodiments, the initial model may be manually input by a user.

The model may include optical properties, such as the scattering coefficient of one or more layers, the absorption coefficient of one or more layers, the angular scattering probabilities of one or more layers, the number of layers of the multilayer environment 101, the thickness of one or more layers, or the refractive index of one or more layers, among other things.

In operation 203, the process 200, using the initial environment model, estimates components of a radiance signal 107 to be measured by the scanner 110 using the initial environmental model. In some embodiments, the scanning system uses a forward model, such as equations (1) and (2), to estimate the radiance signal components 109. For example, the scanning system 100 may use the forward model to determine a source function for the layers of the multilayer environment 101. If the process 200 is being used to compute the signal component of a target layer, the process 200 may determine the source functions for the target layer and the other layers between the target layer and the scanner 110. The scanning system 100 may use the source functions to determine a total radiance reflected back towards the scanner 110. After the source functions are determined, the scanning system 100 determines the signal component for each layer by integrating each source function. In one embodiment, the scanning system 100 determines the signal component for each layer between the target layer and the scanner 110, then determines the target layer signal components by subtracting the total radiance from the signal components for the layers other than the target layer. For example, the scanning system 100 may use equations (1) or (2) to determine the signal component for one or more layers.

In operation 205, the scanner 110 emits the radiance signal 107 into the multilayer environment 101. As the radiance signal 107 passes through each layer of the multilayer environment 101, a portion of the radiance signal is reflected back towards the scanner 110. The radiance signal, altered by the layers of the multilayer environment, is received by the scanner 110 in operation 207. In some embodiments, the scanner 110 emits and receives a continuous radiance signal. In other embodiments, the radiance signal 107 emits and receives a pulsed radiance signal.

After the scanning system 100 receives the radiance signal 107, the scanning system 100 determines the signal component of the radiance signal for one or more of the layers of the multilayer environment 101 in operation 209 using the radiance signal 107 (e.g., by integrating the source functions [see equations (1) and (2)]). In some embodiments, the layer data analysis circuit 121 determines the signal component using equation (1) or (2).

In some embodiments, the process 200 includes comparing the signal components derived in operation 203 with the signal components derived in operation 209. To reduce differences between estimated and actual (e.g., measured) signal components, the scanning system 100 may adjust optical properties of one or more tissue layers of the environmental model. As the optical properties of the model are adjusted, the environmental model begins to reflect the optical properties of the multilayer environment 101.

After determining the radiance signal component 109 for one or more layers of the multilayer environment 101, the scanning system generates an output using the signal component of at least one layer. For example, the output may be a spectral reflectance image visualizing the signal component for a target layer other than the layer closest to the scanner 110, among other things. In another example, the output may include the environmental model with updated optical properties to reflect the optical properties of the multilayer environment. Where the scanner 110 is interacting with an air-tissue multilayer environment, the output may include a form of data which can be used to determine whether tissue is abnormal or healthy.

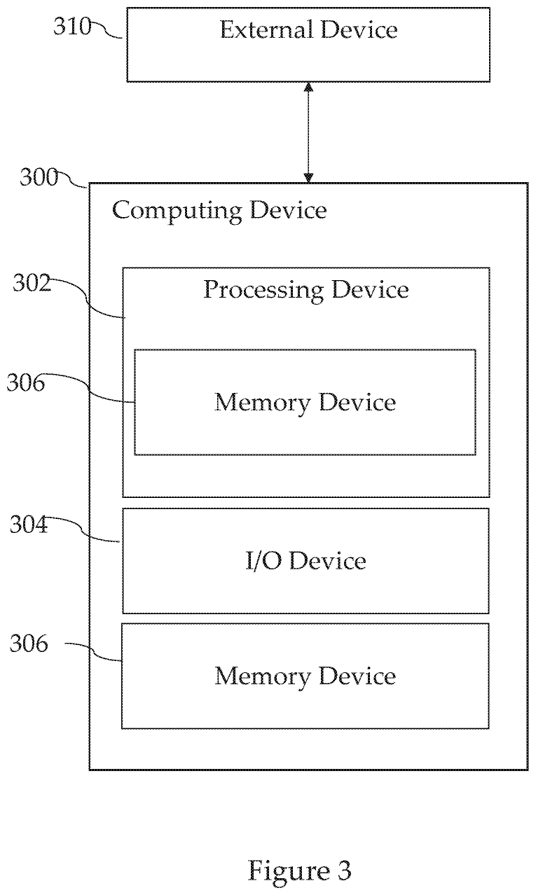

FIG. 3 schematically shows a computing device 300 in accordance with various embodiments. The computing device 300 is one example of a computing device of the scanning system 100, such as the computing system 120, the scanner 110, the measuring circuit 111, the data storage circuit 123, the layer data analysis circuit 121, and the output circuit 125, among other things. The computing device 300 includes a processing device 302, an input/output device 304, and a memory device 306. The computing device 300 may be a stand-alone device, an embedded system, or a plurality of devices configured to perform the functions described with respect to one of the components of scanning system 100. Furthermore, the computing device 300 may communicate with one or more external devices 310.

The input/output device 304 enables the computing device 300 to communicate with an external device 310. For example, the input/output device 304 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 304 may be comprised of hardware, software, or firmware. The input/output device 304 may have more than one of these adapters, credentials, interfaces, or ports, such as a first port for receiving data and a second port for transmitting data, among other things.

The external device 310 may be any type of device that allows data to be input or output from the computing device 300. For example, the external device 310 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 310 may be integrated into the computing device 300. More than one external device may be in communication with the computing device 300.

The processing device 302 may be a programmable type, a dedicated, hardwired state machine, or a combination thereof. The processing device 302 may further include multiple processors, Arithmetic-Logic Units (ALUs), Central Processing Units (CPUs), Digital Signal Processors (DSPs), or Field-programmable Gate Arrays (FPGA), among other things. For forms of the processing device 302 with multiple processing units, distributed, pipelined, or parallel processing may be used. The processing device 302 may be dedicated to performance of just the operations described herein or may be used in one or more additional applications. The processing device 302 may be of a programmable variety that executes processes and processes data in accordance with programming instructions (such as software or firmware) stored in the memory device 306. Alternatively or additionally, programming instructions are at least partially defined by hardwired logic or other hardware. The processing device 302 may be comprised of one or more components of any type suitable to process the signals received from the input/output device 304 or elsewhere, and provide desired output signals. Such components may include digital circuitry, analog circuitry, or a combination thereof.

The memory device 306 in different embodiments may be of one or more types, such as a solid-state variety, electromagnetic variety, optical variety, or a combination of these forms, to name but a few examples. Furthermore, the memory device 306 may be volatile, nonvolatile, transitory, non-transitory or a combination of these types, and some or all of the memory device 306 may be of a portable variety, such as a disk, tape, memory stick, or cartridge, to name but a few examples. In addition, the memory device 306 may store data which is manipulated by the processing device 302, such as data representative of signals received from or sent to the input/output device 304 in addition to or in lieu of storing programming instructions, among other things. As shown in FIG. 3, the memory device 306 may be included with the processing device 302 or coupled to the processing device 302, but need not be included with both.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

What is claimed is:

1. A method for remote sensing of a multilayer environment, comprising:
emitting a radiance signal toward a first layer and a second layer of the multilayer environment;
receiving the radiance signal including a first component corresponding to the first layer, and a second component corresponding to the second layer; and
determining the second component based on the radiance signal,
wherein at least one of the first layer or the second layer is optically thick.

2. The method of claim 1, comprising:
outputting a spectral reflectance image using the determined second component, wherein emitting the radiance signal includes emitting a collimated beam.

3. The method of claim 1, wherein the first layer includes at least one of a different refractive index, a different absorption characteristic, or a different scattering characteristic relative to the second layer.

4. The method of claim 3, wherein the first layer is comprised of gaseous matter, and the second layer is comprised of soft matter.

5. The method of claim 3, wherein a difference between the refractive indices of the first layer and the second layer is at least 0.3.

6. The method of claim 1, wherein determining the second component includes integrating a source function for the first layer.

7. The method of claim 1, further comprising:
estimating radiance signal components using an environment model,
wherein determining the second component includes updating an optical property of the second layer of the multilayer environment after comparing the estimated radiance signal components and the determined second component.

8. A scanning system, comprising:
a measuring circuit structured to emit a radiance signal toward a first layer and second layer of a multilayer environment, and receive the radiance signal including a first component corresponding to the first layer and a second component corresponding to the second layer; and
a layer data analysis circuit configured to determine the second component based on the received radiance signal,
wherein at least one of the first layer or the second layer is optically thick.

9. The scanning system of claim 8, comprising an output circuit configured to output a spectral reflectance image using the determined second component, wherein the measuring circuit emits the radiance signal by emitting a collimated beam.

10. The scanning system of claim 8, wherein the first layer includes at least one of a different refractive index, a different absorption characteristic, or a different scattering characteristic relative to the second layer.

11. The scanning system of claim 10, wherein the first layer is comprised of gaseous matter, and the second layer is comprised of soft matter.

12. The scanning system of claim 10, wherein a difference between the refractive indices of the first layer and the second layer is at least 0.3.

13. The scanning system of claim 8, wherein determining the second component includes integrating a source function.

14. The scanning system of claim 8, wherein the data layer analysis circuit is configured to estimate radiance signal components using an environment model,
wherein determining the second component includes updating an optical property of the first layer and the second layer of the multilayer environment after comparing the estimated radiance signal components with the determined first and second components.

15. A computer program product for remote sensing of a multilayer environment, the computer program product comprising a tangible, non-transient computer usable medium having computer readable program code thereon, the computer readable program code comprising:
program code for emitting a radiance signal through a first layer and then a second layer of the multilayer environment, at least one of the first layer or the second layer being optically thick;
program code for receiving radiance signal components including a first component corresponding to the first layer and a second component corresponding to the second layer; and
program code for determining the second component based on the radiance signal.

16. The computer program product of claim 15, comprising:
outputting a spectral reflectance image using the determined second component, wherein emitting the radiance signal includes emitting a collimated beam.

17. The computer program product of claim 15, wherein the first layer includes at least one of a different refractive index, a different absorption characteristic, or a different scattering characteristic relative to the second layer.

18. The computer program product of claim 17, wherein a difference between the refractive indices of the first layer and the second layer is at least 0.3.

19. The computer program product of claim 15, wherein determining the second component includes integrating a source function.

20. The computer program product of claim 15, further comprising:
program code for estimating radiance signal components using an environment model,
wherein determining the second component includes updating an optical property of the second layer of the multilayer environment after comparing the estimated radiance signal components with the corresponding determined second component.
